(19) United States
(12) Plant Patent Application Publication
(10) Pub. No.: US 2002/0148021 P1
(43) Pub. Date: Oct. 10, 2002

(54) Asclepias plant named 'Red Wijna'

(76) Inventors: Frank Stricker, Rijsenhout (NL); Jacqueline van Klingeren, Rijsenhout (NL)

Correspondence Address: Law Offices, FOLEY & LARDNER, Suite 500, 3000 K Street, N.W., Washington, DC, 20007-5109, US

(21) Appl. No.: 09/768,229

(22) Filed: Jan. 25, 2001

(51) Int. Cl. A01H 5/00

(52) U.S. Cl. PLT/263

(57) ABSTRACT

A new and distinct Asclepias plant named ‘Red Wijna’ characterized by having long stem length approximately 80 cm when grown in the open, red/orange flower color, lance shaped to oblong leaves up to 4 cm wide, and very high temperature tolerance.

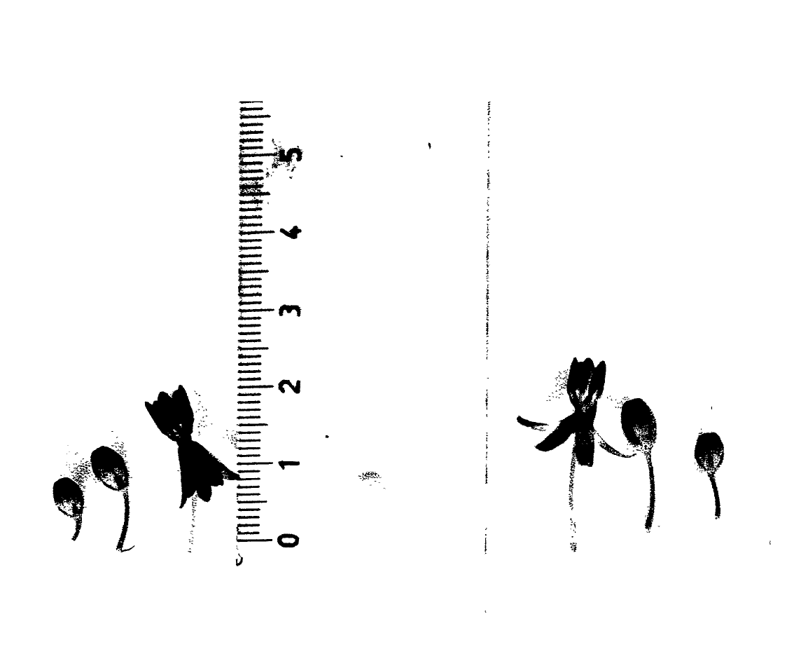

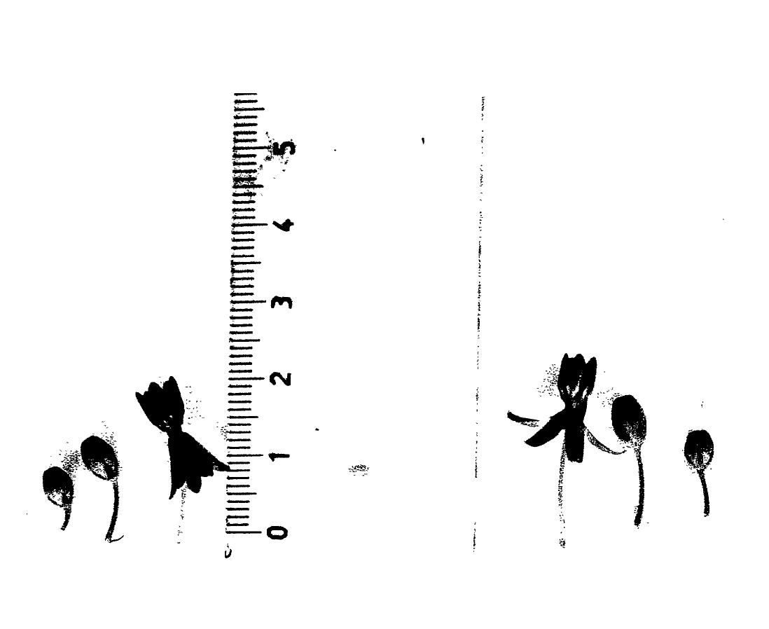

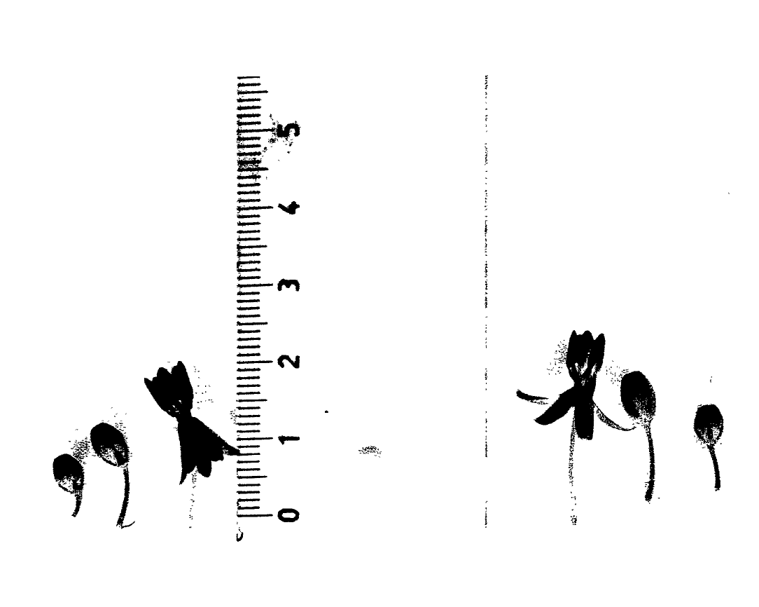

### BACKGROUND OF THE INVENTION

The present invention relates to a new and distinct cultivar of Asclepias plant, hereinafter referred to by the cultivar name ‘Red Wijna’.

The genus Asclepias is a member of the milkweed family (Asclepiadaceae). There are approximately 200 species in the genus Asclepias found principally in North America and Africa. The genus Asclepias is also known as milkweed, silkweed or butterfly flower. Members of the genus Asclepias are mostly perennial herbs that generally produce milky sap.

The new cultivar was originated from a cross made in a uncontrolled breeding program in Amsterdam, The Netherlands. The female parent is Asclepias tuberosa ‘Gay Butterfly’. The male parent is Asclepias tuberosa. ‘Red Wijna’was discovered and selected by the inventors, Frank Stricker and Jacqueline van Klingeren, as a flowering plant within the progeny of the stated cross in a controlled environment in Amsterdam, The Netherlands.

Asexual reproduction of the new cultivar by tissue culture was first performed in the Fall of 1998 in Rhenen and has demonstrated that the combination of characteristics as herein disclosed for the new cultivar are firmly fixed and retained through successive generations of asexual reproduction.

### BRIEF DESCRIPTION OF THE INVENTION

The following traits have been repeatedly observed and are determined to be basic characteristics of ‘Red Wijna’ which in combination distinguish this Asclepias as a new and distinct cultivar:

1. long stem length, approximately 80 cm when grown in the open;

2. red/orange flower color;

3. peduncle length between 20 to 25 mm;

4. style color is light green (RHS 146 D), ovary color is a little lighter; and

5. very high temperature tolerance;

6. some parts of the green stem (RHS 146 C fading to 146 D on top) are light brown (RHS 200D); and

7. lance-shaped to oblong leaves up to 4 cm wide.

‘Red Wijna’ has not been observed under all possible environmental conditions. The phenotype of the new cultivar may vary significantly with variations in environment such as temperature, light intensity, and day length without any change in the genotype of the plant. The following observations, measurements and values describe the new cultivar as grown in Haarlem, The Netherlands under conditions which closely approximate those generally used in commercial practice.

Of the many commercial cultivars known to the present inventor, the most similar in comparison to ‘Red Wijna’ is the cultivar ‘Tuberosa’. The flowers of ‘Red Wijna’ are more red than orange, whereas tuberosa is orange. The stem length of ‘Red Wijna’ is approximately 5 to 10 cm longer than the stem length of ‘Tuberosa’. The peduncle length of ‘Red Wijna’ is slightly larger than that of ‘Tuberosa’. The leaves of ‘Red Wijna’ are much wider than the leaves of ‘Tuberosa’. The color of young leaves of ‘Red Wijna’ is darker than the leaves of ‘Tuberosa’.

| | ‘Red Wijna’ | ‘Tuberosa’ |
|---|---|---|
| Upper side | Green RHS 137 A | Closest to Green RHS 137 |
| young leaf | | B to 137 C |
| Under side | Green RHS 137 C | Between RHS 137 C and 137 D |
| young leaf | | |
| Upper side | Closest to green RHS 137 | Closest to RHS 137 C but darker |
| adult leaf | A but darker than young | than young leaf |
| Under side | Green RHS 137 C | Green between RHS 137 C and |
| adult leaf | but darker than young leaf | D but darker than young leaf |

### BRIEF DESCRIPTION OF THE DRAWING

The accompanying photographic illustration shows typical flower and foliage characteristics of ‘Red Wijna’ with colors being as true as possible with illustrations of this type.

### DETAILED BOTANICAL DESCRIPTION

The following observations, measurements and values describe the new cultivar as grown in Haarlem, The Netherlands under conditions which closely approximate those generally used in commercial practice. ‘Red Wijna’ is grown in a normal greenhouse or in open ground.

When grown in a green house in The Netherlands, ‘Red Wijna’ is harvested three times per year under temperature and photoperiodic controlled conditions. The first stems are harvested in May, the last of the third harvest in November. When grown outdoor, only one harvest is possible and only in the summer.

‘Red Wijna’ has a very high temperature tolerance of more than 50 degrees celsius as measured in the inventor's greenhouse on warm, sunny summer days. The inventor keeps day and night temperature always above 18 degrees celcius. ‘Red Wijna’ is tolerant to frost but below 9 degrees celsius, ‘Red Wijna’ becomes dormant.

The inventor gives ‘Red Wijna’ 20 hours of light from February, when the breeding starts until the end of November when the last stems are harvested. ‘Red Wijna’ is sensitive to Rhizoctonia, thrip, spider mite and lice, and to a lesser extent, mildew. No fertilizers are added to the ground.

Color references are made to The Royal Horticultural Society Colour Chart (RHS), except where general colors of ordinary significance are used. Color values were taken under daylight conditions at approximately noon in Rijswijk, The Netherlands.

Parentage:

Male parent.—Asclepias tuberosa ‘Gay Butterfly’.

Female parent.—Asclepias tuberosa ‘Red Wijna’ is a selection from seedlings of the above-mentioned Asclepias varieties.

Classification:

Botanical.—Asclepias.

Commercial.—Butterfly cultivar ‘Red Wijna’.

Propagation: Tissue culture

Plant:

Growth habit.—Upright, vertical.

Foliage:

Shape.—Lance shaped to oblong, with pubescence on underside.

Color.—Green RHS 137.

Size.—Approximately 11 cm long, approximately 4 cm wide.

Orientation.—Alternate on three sides of the stem.

Inflorescence:

Umbel.—Up to 10 per stem in up to 4 branches on top of the stem, up to 19 flowers per umbel, up to 150 per stem. The oldest umbel is approximately 30-35 mm high and is 60-70 mm in diameter, younger umbels are smaller.

Pedicel length.—20-25 mm.

Pedicel color.—Green, RHS 146 D.

Flowers:

Bisexual.—Yes.

Blooming Period.—May to November in greenhouse, outdoor only in summer

Flowering Stem.—Length is approximately 80 cm, no lateral branches, strong stem with pubescence, color is RHS 146 C (stem bottom), fades to 146 D, parts of the top are light brown RHS 200 D

Corolla Length.—Up to 8 mm long, 6-7 mm diameter Color: Orange/red RHS 34 B Lobes: 5

Stamen.—Fused Number: 5 Color: Orange/red RHS 34 B

Seeds.—Present Color: Dark brown housed in green fruits Hairs: Tuft of long silky white hairs

I claim:

1. A new and distinct Asclepias plant named ‘Red Wijna’, substantially as illustrated and described herein.
